(19) United States
(12) Patent Application Publication
(10) Pub. No.: US 2021/0386843 A1
(43) Pub. Date: Dec. 16, 2021

(54) mRNA Vaccine

(71) Applicant: eTheRNA Immunotherapies NV, Niel (BE)

(72) Inventors: Stefaan De Koker, Niel (BE); Lukasz Bialkowski, Niel (BE)

(21) Appl. No.: 17/420,556

(22) PCT Filed: Jan. 3, 2020
(86) PCT No.: PCT/EP2020/050072

§ 371 Date: Jul. 2, 2021

(30) Foreign Application Priority Data
Jan. 4, 2019 (EP) 19150349.9
Mar. 18, 2019 (EP) 19163486.4

(51) Int. Cl. A61K 39/00; C07K 16/28; A61K 39/395; A61P 35/00

(52) U.S. Cl. CPC A61K 39/0011; C07K 16/2818; A61K 39/3955; A61K 39/0005; A61P 35/00; A61K 2039/54; C07K 2317/76; A61K 2039/505; A61K 2039/53; A61K 2039/6031; A61K 2039/55555; C07K 2317/569

(57) ABSTRACT

The present invention in general relates to a combination of mRNA molecules encoding functional immunostimulatory proteins and a PD-1 pathway inhibitor. In particular, it relates to a combination of one or more mRNA molecules encoding at least one functional immunostimulatory protein selected from the list comprising: CD40L, CD70 and caTLR4; and a PD-1 pathway inhibitor, optionally also in the form of an mRNA molecule. The present invention further relates to vaccines comprising such combination, as well as uses of the combinations and vaccine of the present invention in human or veterinary medicine, in particular in the prevention and/or treatment of cell proliferative disorders.

### FIELD OF THE INVENTION

The present invention in general relates to a combination of mRNA molecules encoding functional immunostimulatory proteins and a PD-1 pathway inhibitor. In particular, it relates to a combination of one or more mRNA molecules encoding at least one functional immunostimulatory protein selected from the list comprising: CD40L, CD70 and caTLR4; and a PD-1 pathway inhibitor, optionally also in the form of an mRNA molecule. The present invention further relates to vaccines comprising such combination, as well as uses of the combinations and vaccine of the present invention in human or veterinary medicine, in particular in the prevention and/or treatment of cell proliferative disorders.

### BACKGROUND TO THE INVENTION

The induction of potent cytolytic CD8 T cell responses capable of recognizing and killing cancer cells constitutes the key goal of any therapeutic cancer vaccine. The capacity of a vaccine to elicit such cytolytic T cell responses is heavily determined by the early interaction between the vaccine and dendritic cells (DCs), the most potent antigen presenting cells and instigators of T cell immunity. In contrast to protein-based vaccines, mRNA vaccines enable expression of the mRNA encoded antigen in the cytosol of DCs, the natural route of antigen processing and presentation of antigens to CD8 T cells.

In addition, in vitro transcribed mRNA—produced by viral polymerases such as T7—partially resembles a viral RNA, and is hence recognized by innate immune sensors, endowing the mRNA with intrinsic adjuvant properties (Kariko et al., 2005; Yoneyama et al., 2010). Nonetheless, activation of DCs by IVT mRNA is suboptimal, and can be further enhanced by the co-delivery of TriMix mRNA, a mix of three mRNAs encoding the immune-stimulatory proteins CD40L, CD70 and caTLR4 (Bonehill et al., 2008; Van Lint et al. 2012; Van Lint et al., 2016). Addition of TriMix mRNA to mRNA encoding tumor antigens has been demonstrated to strongly enhance the magnitude of the T cell response and its antitumor efficacy in preclinical models and is currently explored in clinical studies. Hence, the inventors have previously established that the T cell stimulatory capacity of APCs can be greatly enhanced by providing them with specific molecular adjuvants in the form of a mixture of mRNA or DNA molecules encoding one or more of the immunostimulatory factors CD40L, CD70 and caTLR4, either in vivo or in vitro. Said stimulation with immunostimulatory factors can be done in vivo (in situ) through intravenous, intratumoral, intradermal, intraperitoneal, intramuscular or intranodal administration of mRNA or DNA molecules encoding said one or more immunostimulatory factors and optionally a tumor antigen mRNA, DNA or protein. Said mRNA or DNA can be naked or can be protected as described below. Said mRNA or DNA can be protected when administered for example intravenously.

The PD-1 gene, which belongs to the immunoglobulin super family, encodes a 55 kDa type I transmembrane protein. Both mouse PD-1 and human PD-1 consist of 288 amino acids, and have a signal peptide at the N terminal (20 amino acid) and hydrophobic region in the middle part, which is a transmembrane region.

In thymus, PD-1 is expressed at the transition phase between CD4−/CD8− to CD4+/CD8+stage on thymocytes. In periphery, PD-1 is expressed on T cells and B cells activated through the antigen receptor, and on activated myeloid lineage cells such as macrophages.

PD-1 has an ITIM (Immunoreceptor tyrosine-based inhibitory motif) in its intracellular region, and therefore, PD-1 is considered a negative regulator in immune responses.

PD-1 inhibitors have been approved such as Nivolumab and Pembrolizumab, which prevent the inhibitory signals between PD-1 and PD-L1. While these drugs have potentiated durable responses in some patients, the response rates of these drugs as monotherapy have been low. In particular, these drugs are faced with a lack of clinical benefit in most patients, since success of the therapy depends heavily on the presence of pre-existing anti-tumor T cells in the tumor bed. Moreover, T cell exhaustion or paralysation of said anti-tumor T cells often occurs in the context of therapeutic cancer vaccines. Specifically, intratumor immunomodulators can initiate/amplify the tumor-specific T cell responses, but often these T cells are “paralyzed” by the immunosuppressive tumor micro-environment; which amongst others occurs via PD1 signalling. Hence, it was an object of the present invention to provide a more potent anti-cancer composition based on PD-1 inhibitors. We have now surprisingly found that the combination of PD-1 inhibitors and the immunostimulatory factors of the present invention (CD40L, CD70 and/or caTLR4), result in a significant immunostimulatory effect, rendering said combinations highly suitable in the context of anti-cancer vaccination and treatment.

### SUMMARY OF THE INVENTION

The present invention is defined by the following numbered statements:

1. A combination comprising:

one or more mRNA molecules encoding at least one functional immunostimulatory protein selected from the list comprising: CD40L, CD70 and caTLR4; and

a PD-1 pathway inhibitor, which in particular prevents or blocks PD-1 initiated signalling.

2. The combination as defined in statement 1; wherein said one or more mRNA molecules encode all of the functional immunostimulatory proteins selected from the list comprising:

CD40L, CD70 and caTLR4.

3. The combination as defined in statement 1; wherein said PD-1 pathway inhibitor is in the form of mRNA encoding said PD-1 pathway inhibitor.

4. The combination as defined in statement 1; wherein said PD-1 pathway inhibitor is selected from the list comprising: a nanobody directed against PD-1, an antagonistic antibody directed against PD-1; a nanobody directed against PDL1, an antagonistic antibody directed against PDL1; or a derivative thereof; more in particular an antagonistic antibody directed against PD-1.

5. The combination as defined in statement 4; wherein said antagonistic antibody directed against PD-1 is selected from the list comprising: nivolumab (BMS-936558/MDX1106), pidilizumab (CT-011), pembrolizumab (MK-3475).

6. The combination as defined in anyone of statements 1-5; further comprising one or more mRNA molecules encoding a target-specific antigen; more in particular a tumor-associated antigen.

7. The combination as defined in statement 6; wherein said (mRNA molecules encoding a) target-specific antigen is selected from the list comprising: total mRNA isolated from (a) target cell(s), one or more target-specific mRNA molecules, protein lysates of (a) target cell(s), specific proteins from (a) target cell(s), a synthetic target-specific peptide or protein, and synthetic mRNA or DNA encoding a target-specific antigen or its derived peptide(s).

8. The combination as defined in statement 7; wherein said target-specific antigen is a tumor antigen.

9. The combination as defined in anyone of statements 1-3; wherein said one or more mRNA molecules are formulated for parenteral administration; more in particular for intravenous, intratumoral, intradermal, subcutaneous, intraperitoneal, intramuscular or intranodal administration.

10. The combination as defined in anyone of statements 1-3; wherein said mRNA molecules are formulated for intravenous administration, and encompassed in nanoparticles, such as polymeric or lipid nanoparticles.

11. The combination as defined in anyone of statements 1-3; wherein said mRNA molecules are formulated for intratumoral or intranodal administration, and are in the form of naked mRNA molecules in a suitable injection buffer, such as a Ringer Lactate buffer.

12. The combination as defined in anyone of statements 1-3; wherein said PD-1 pathway inhibitor is formulated for parenteral administration; more in particular for intravenous, intratumoral, intradermal, subcutaneous, intraperitoneal, intramuscular or intranodal administration.

13. A vaccine comprising the combination as defined in anyone of statements 1-12.

14. The vaccine as defined in statement 12: wherein said vaccine is formulated for parenteral administration; more in particular for intravenous, intratumoral, intradermal, subcutaneous, intraperitoneal, intramuscular or intranodal administration.

15. The combination as defined in anyone of statements 1-12 or the vaccine as defined in anyone of statements 13-14 for use in human or veterinary medicine.

16. The combination as defined in anyone of statements 1-12 or the vaccine as defined in anyone of statements 13-14 for use in the treatment of cell proliferative disorders.

17. The combination as defined in anyone of statements 1-12 or the vaccine as defined in anyone of statements 13-14 for use in eliciting an immune response towards a tumor in a subject.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Illustration of possible routes of administration in the context of the invention.

FIG. 2: Effect of combination of IN administered Trimix and antigen with IP administered anti-PD-1 on tumor growth.

### DETAILED DESCRIPTION OF THE INVENTION

As already detailed herein above, the present invention relates to a combination comprising:

one or more mRNA molecules encoding at least one functional immunostimulatory protein selected from the list comprising: CD40L, CD70 and caTLR4; and

a PD-1 pathway inhibitor; in particular, a PD-1 pathway inhibitor which prevents or blocks PD-1 initiated signalling.

Throughout the invention, the term “TriMix” stands for a mixture of mRNA molecules encoding CD40L, CD70 and caTLR4 immunostimulatory proteins.

The mRNA or DNA used or mentioned herein can either be naked mRNA or DNA, or protected mRNA or DNA. Protection of DNA or mRNA increases its stability, yet preserving the ability to use the mRNA or DNA for vaccination purposes. Non-limiting examples of protection of both mRNA and DNA can be: liposome-encapsulation, protamine-protection, (Cationic) Lipid Lipoplexation, lipidic, cationic or polycationic compositions, Mannosylated Lipoplexation, Bubble Liposomation, Polyethylenimine (PEI) protection, liposome-loaded microbubble protection etc. The term “target” used throughout the description is not limited to the specific examples that may be described herein. Any infectious agent such as a virus, a bacterium or a fungus may be targeted. In addition any tumor or cancer cell may be targeted.

The term “target-specific antigen” used throughout the description is not limited to the specific examples that may be described herein. It will be clear to the skilled person that the invention is related to the induction of immunostimulation in APCs, regardless of the target-specific antigen that is presented. The antigen that is to be presented will depend on the type of target to which one intends to elicit an immune response in a subject. Typical examples of target-specific antigens are expressed or secreted markers that are specific to tumor, bacterial and fungal cells or to specific viral proteins or viral structures. Without wanting to limit the scope of protection of the invention, some examples of possible markers are listed below.

The term “antigen presenting cell” used throughout the description includes all APCs. Specific non limiting examples are DCs, dendritic cell-lines, B-cells, or B-cell-lines. The DCs or B-cells can be isolated or generated from the blood of a patient or healthy subject. The patient or subject can have been the subject of prior vaccination or not.

The terms “neoplasms”, “cancer” and/or “tumor” used throughout the description are not intended to be limited to the types of cancer or tumors that may have been exemplified. The term therefore encompasses all proliferative disorders such as neoplasma, dysplasia, premalignant or precancerous lesions, abnormal cell growths, benign tumors, malignant tumors, cancer or metastasis, wherein the cancer is selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma. Specific antigens for cancer can e.g. be MelanA/MART1, Cancer-germline antigens, gp100, Tyrosinase, CEA, PSA, Her-2/neu, survivin, telomerase.

The use of the combination of CD40L and caTLR4 generates mature, cytokine/chemokine secreting DCs, as has been shown for CD40 and TLR4 ligation through addition of soluble CD40L and LPS.

The introduction of CD70 into the DCs provides a co-stimulatory signal to CD27⁺ naive T-cells by inhibiting activated T-cell apoptosis and by supporting T-cell proliferation.

As an alternative to caTLR4, other Toll-Like Receptors (TLR) could be used. For each TLR, a constitutive active form is known, and could possibly be introduced into the DCs in order to elicit a host immune response. In our view however, caTLR4 is the most potent activating molecule and is therefore preferred.

In a preferred embodiment of the vaccine of the invention, the mRNA or DNA molecule(s) encode(s) the CD40L and CD70 immunostimulatory proteins. In a particularly preferred embodiment of the vaccine of the invention, the mRNA or DNA molecule(s) encode(s) CD40L, CD70, and caTLR4 immunostimulatory proteins.

Said mRNA or DNA molecules encoding the immunostimulatory proteins can be part of a single mRNA or DNA molecule. Preferably, said single mRNA or DNA molecule is capable of expressing the two or more proteins simultaneously. In one embodiment, the mRNA or DNA molecules encoding the immunostimulatory proteins are separated in the single mRNA or DNA molecule by an internal ribosomal entry site (IRES) or a self-cleaving 2a peptide encoding sequence.

In a specific embodiment, one or more of said mRNA molecules of the present invention may further contain a translation enhancer and/or a nuclear retention element. Suitable translation enhancers and nuclear retention elements are those described in WO2015071295.

As used herein, the term “PD-1 inhibitor” includes any compound able to directly or indirectly affect the regulation of PD-1 by reducing for example the expression of PD-1 (i.e., transcription and/or the translation) or its natural ligands PD-L1/PD-L2, or a PD-1 activity. It includes intracellular (e.g., agents that block a PD-1-associated signalling molecule or pathway, such as SHP-1 and SHP-2) as well as extracellular PD-1 inhibitor. Without being so limited, such inhibitors include siRNA, antisense molecules, proteins, peptides, small molecules, antibodies, etc.

Hence, in a specific embodiment, said PD-1 pathway inhibitor is selected from the list comprising: a nanobody directed against PD-1, an antagonistic antibody directed against PD-1; a nanobody directed against PDL1, an antagonistic antibody directed against PDL1; or a derivative thereof Derivatives of antibodies may for example include scFV, bispecific antibodies.

In an embodiment, the above-mentioned PD-1 inhibitor blocks/inhibits the interaction between PD-1 and a PD-1 ligand (e.g., PD-L1, PD-L2). Such inhibitor may target, for example, the IgV domain of PD-1 and/or PD-L1 and/or PD-L2, such as one or more of the residues involved in the interaction, as discussed above.

In an embodiment, the above-mentioned PD-1 inhibitor is a blocking antibody, such as an anti-PD-1 or anti-PD-L1/PD-L2 antibody. Blocking anti-PD-1 and/or anti-PD-L1/PD-L2 antibodies are well known in the art. Other blocking antibodies may be readily identified and prepared by the skilled person based on the known domain of interaction between PD-1 and PD-L1/PD-L2, as discussed above. For example, a peptide corresponding to the IgV region of PD-1 or PD-L1/PD-L2 (or to a portion of this region) could be used as an antigen to develop blocking antibodies using methods well known in the art.

By “anti-PD-1 antibody” or “anti-PD-L1” or “anti-PD-L2” in the present context is meant an antibody capable of detecting/recognizing (i.e. binding to) a PD-1, PD-L1 or PD-L2 protein or a PD-1, PD-L1 or PD-L2 protein fragment. In an embodiment, the above-mentioned antibody inhibits the biological activity of PD-1, such as PD-1-PD-L1/PD-L2 interaction or PD-1-mediated T cell inhibition. In another embodiment, the PD-1 or PD-L1/PD-L2 protein fragment is an extracellular domain of PD-1 or PD-L1/PD-L2 (e.g., the IgV domain).

The term “PD-1 levels” as used herein means the level of expression of PD-1 on T cells or the percent of PD-1 positive T cells that are also CD4+ or CD8+ expressed as a percent of the total number of CD4+ or CD8+ cells. The level of expression of PD-1 on T cells can be determined by any standard method. For example, it can be determined by flow cytometry and measured as mean fluorescence intensity (MFI).

In one embodiment, the disclosure provides a method for a combination therapy for individuals comprising administering to the individual one or more immunostimulatory factors and an inhibitor of PD-1. The one or more immunostimulatory factors and the PD-1 inhibitor may be administered simultaneously or contemporaneously, as a single composition or separate compositions, or the one or more immunostimulatory factors and PD-1 inhibitor may be administered at different times.

The compositions of the present invention generally include a PD-1 inhibitor in combination with one or more immunostimulatory factors. PD-1 is a cell surface receptor that belongs to the immunoglobulin superfamily and is expressed on T cells and pro-B cells. PD-1 binds two ligands, PD-L1 and PD-L2. PD-1, plays an important role in down regulating the immune system by preventing the activation of T-cells, which in turn reduces autoimmunity and promotes self-tolerance. The inhibitory effect of PD-1 is accomplished through a dual mechanism of promoting apoptosis (programmed cell death) in antigen specific T-cells in lymph nodes while simultaneously reducing apoptosis in Tregs. Suitable PD-1 inhibitor for use in the compositions of the present disclosure include nivolumab (BMS-936558/MDX1106), pidilizumab (CT-011), pembrolizumab (MK-3475), and combinations thereof; alternatively the PD-L1 inhibitor atezolizumab may also be suitably used within the context of the invention.

Suitable dosages of the PD-1 inhibitor will depend upon a number of factors including, for example, age and weight of an individual, at least one precise condition requiring treatment, severity of a condition, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

The present invention also provides a combination as defined herein; wherein said mRNA molecules are formulated for intravenous administration, and encompassed in (lipid) nanoparticles. A lipid nanoparticle (LNP) is generally known as a nanosized particle composed of a combination 25 of different lipids. While many different types of lipids may be included in such LNP, the LNP's of the present invention may for example be composed of a combination of an ionisable lipid, a phospholipid, a sterol and a PEG lipid.

As used herein, the term “nanoparticle” refers to any particle having a diameter making the 30 particle suitable for systemic, in particular intravenous administration, of, in particular, nucleic acids, typically having a diameter of less than 1000 nanometers (nm).

In an alternative embodiment, the present invention provides a combination as defined herein; wherein said mRNA molecules are formulated for intratumoral or intranodal administration, and are in the form of naked mRNA molecules in a suitable injection buffer, such as a Ringer Lactate buffer. The present invention also provides the combinations and vaccines as defined herein for use in human or veterinary medicine, in particular for use in the treatment of cell proliferative disorders, more in particular for use in eliciting an immune response towards a tumor in a subject.

Finally, the present invention provides a method for the treatment of a cell proliferative disorder comprising the steps of administering to a subject in need thereof a combination or vaccine of the present invention.

### EXAMPLES

Example 1: Combination of Trimix, antigen and anti-PD-1.

The scope of this example is to elicit the T-cell immune responses against pre-defined TAAs when using Trimix either as a monotherapy or in combination with anti-PD-1. The advantages of this setting include:

The ability to incorporate TAAs

Trimix mRNA acts as an adjuvant to augment T-cell responses against TAA

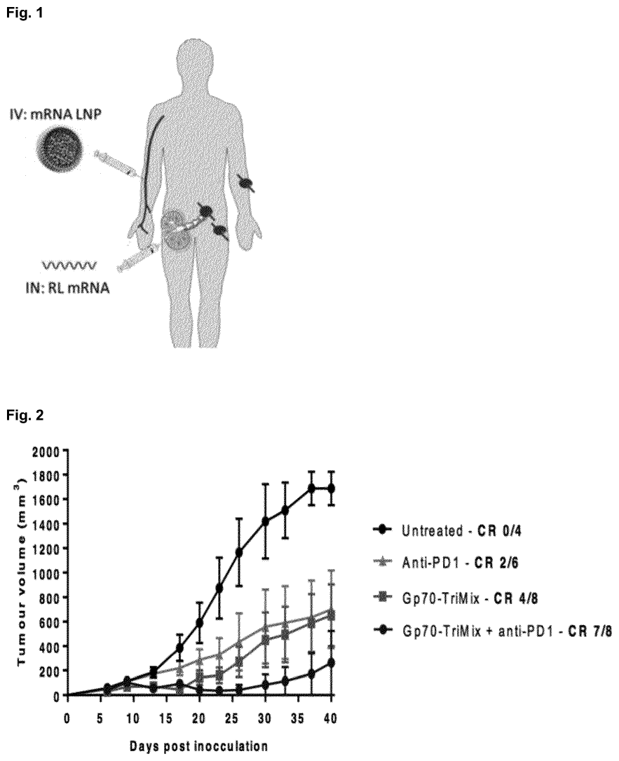

Two routes of administration are explored (intranodal (IN) and intravenous (IV)), such as illustrated in FIG. 1

IV administration was shown to be more immunogenic, however, also necessitates additional formulation, such as using LNPs.

Balb/C female mice were injected with 1×10⁶ CT26 tumor cells on day 0. At days 5, 10, and 15 GP70-Trimix mRNA (10 μg per component) was intranodally administered. Monoclonal anti-PD-1 antibodies (10 mg/kg) were intraperitoneally administered at day 5, and repeated in total 5 times with intervals of 3 days. Complete responders (CR) were considered if being tumor free at day 40.

The results of this example are shown in FIG. 2; which clearly demonstrates a beneficial effect of both anti-PD-1 (complete responders 2 out of 6) and GP70-Trimix (complete responders 4 out of 8) on tumor growth, when used alone. Moreover, the combined use of GP70-Trimix and anti-PD-1 show an even further increased effect on tumor growth resulting in 7 out of 8 mice being completely tumor free at day 40.

1-13. (canceled)

14. A combination comprising:
one or more mRNA molecules encoding all of the functional immunostimulatory proteins CD40L, CD70 and constitutive active TLR4 (caTLR4);
one or more mRNA molecules encoding a target-specific antigen; and
a PD-1 pathway inhibitor selected from a nanobody directed against PD-1, an antagonistic antibody directed against PD-1, a nanobody directed against PDL1, an antagonistic antibody directed against PDL1, or a derivative thereof, wherein the one or more mRNA molecules are formulated for intravenous administration.

15. The combination of claim 14, wherein the PD-1 pathway inhibitor is an mRNA encoding the PD-1 pathway inhibitor.

16. The combination of claim 14, wherein the antagonistic antibody directed against PD-1 is selected from the group consisting of nivolumab (BMS-936558/MDX1106), pidilizumab (CT-011), and pembrolizumab (MK-3475).

17. The combination of claim 14, wherein the target-specific antigen is a marker specific to a tumor, a bacterial cell, a fungal cell, a viral protein, or a viral structure.

18. The combination of claim 14, wherein the target-specific antigen is a tumor-associated antigen.

19. The combination of claim 14, wherein the mRNA molecules are formulated for intravenous administration, and encompassed in nanoparticles.

20. The combination of claim 19, wherein the nanoparticles are lipid nanoparticles.

21. The combination of claim 20, wherein the lipid nanoparticles comprise a combination of an ionizable lipid, a phospholipid, a sterol, and a PEG lipid.

22. The combination of claim 14, wherein the PD-1 pathway inhibitor is formulated for parenteral administration.

23. The combination of claim 14, wherein the PD-1 pathway inhibitor is formulated for intravenous administration, intratumoral administration, intradermal administration, sub cutaneous administration, intraperitoneal administration, intramuscular administration, or intranodal administration.

24. A vaccine comprising the combination of claim 14.

25. The vaccine of claim 24, wherein the vaccine is formulated for parenteral administration.

26. The vaccine of claim 24, wherein the vaccine is formulated for intravenous administration, intratumoral administration, intradermal administration, subcutaneous administration, intraperitoneal administration, intramuscular administration, or intranodal administration.

27. A human or veterinary medicine comprising the combination of claim 14.

28. A human or veterinary medicine comprising the vaccine of claim 24.

29. A method for treating a cell proliferative disorder, the method comprising administering the combination of claim 14 to a subject in need thereof.

30. A method for treating a cell proliferative disorder, the method comprising administering the vaccine of claim 24 to a subject in need thereof.

31. A method for eliciting an immune response toward a tumor in a subject, the method comprising administering the combination of claim 14 to a subject in need thereof

32. A method for eliciting an immune response toward a tumor in a subject, the method comprising administering the vaccine of claim 24 to a subject in need thereof.
